(19) United States
(12) Patent Application Publication
(10) Pub. No.: US 2024/0398377 A1
(43) Pub. Date: Dec. 5, 2024

(54) MEDICAL DEVICE

(71) Applicant: FUJIFILM Healthcare Corporation, Chiba (JP)

(72) Inventor: Yuta Tokita, Chiba (JP)

(73) Assignee: FUJIFILM Healthcare Corporation, Chiba (JP)

(21) Appl. No.: 18/661,686

(22) Filed: May 12, 2024

(30) Foreign Application Priority Data
May 29, 2023 (JP) 2023-087750

(51) Int. Cl. A61B 8/00; A61B 50/13; B60B 33/00

(52) U.S. Cl. CPC A61B 8/4405; A61B 50/13; B60B 33/0078

(57) ABSTRACT

An ultrasound diagnostic apparatus includes: a plurality of drive casters; a plurality of lock mechanisms that are respectively provided in the plurality of drive casters, each of the plurality of lock mechanisms locking movement of a corresponding drive caster; and an indicator that presents whether the lock mechanism is in a locked state in which the movement of the drive caster is locked or in a released state in which the movement of the drive caster is allowed.

### CROSS-REFERENCE TO RELATED APPLICATION

This application claims the priority benefit of Japan application serial no. 2023-087750, filed on May 29, 2023. The entirety of the above-mentioned patent application is hereby incorporated by reference herein and made a part of this specification.

### BACKGROUND

### 1. Technical Field

The present disclosure relates to a movable medical device having a plurality of drive casters.

### 2. Description of the Related Art

In the related art, a movable medical device having a plurality of drive casters is known. For example, JP5385721B discloses a movable ultrasound diagnostic apparatus having a plurality of drive casters. The ultrasound diagnostic apparatus of JP5385721B comprises a lock mechanism that switches between a state in which the drive caster is locked and a state in which the lock is released. By providing such a lock mechanism, it is possible to effectively prevent unintentional movement of the ultrasound diagnostic apparatus.

### SUMMARY OF THE INVENTION

However, the medical device in the related art such as JP5385721B does not have a function of presenting the state of the lock mechanism to a user. As a result, in the medical device in the related art, the state of the lock mechanism is mistakenly recognized. For example, in the medical device in the related art, the user mistakenly recognizes that the lock mechanism is locked even though the lock is released.

Of course, even in the medical device in the related art, the user can understand the state of the lock mechanism by checking a state of a member (for example, a pedal) related to the lock mechanism. For example, in a case of the ultrasound diagnostic apparatus of JP5385721B, the state of the lock mechanism can be understood to some extent by checking an inclined angle of the pedal to be stepped on in order to switch the state of the lock mechanism. However, in general, it is difficult for the user to intuitively understand the state of the lock mechanism from the inclined angle of the pedal. Therefore, in the medical device in the related art, it was difficult to easily check the state of the lock mechanism.

Therefore, the present disclosure is directed to a medical device that enables a user to more easily understand whether drive caster is in a locked state or in a released state.

A medical device of the present disclosure comprises: a plurality of drive casters; a plurality of lock mechanisms that are respectively provided in the plurality of drive casters, each of the plurality of lock mechanisms locking movement of a corresponding drive caster; and an indicator that presents whether the lock mechanism is in a locked state in which the movement of the drive caster is locked or in a released state in which the movement of the drive caster is allowed.

With such a configuration, the user can easily understand the state of the lock mechanism.

In this case, the indicator may have a display area in which the state of the lock mechanism is displayed, and the display area may be disposed upward or disposed in a posture obliquely facing an upper front direction toward a front upper side, at a lower front part of the medical device.

With such a configuration, in a case where the user pays attention to the drive casters or the switching pedal, the indicator can be naturally visible to the user.

In addition, the medical device may further comprise: a rotating shaft that switches the state of the lock mechanism by rotating, and the display area may be disposed within a width of the rotating shaft.

With such a configuration, a configuration of a mechanical element that mechanically transmits the movement of the rotating shaft to a constituent element of the indicator can be simplified.

In addition, the medical device may further comprise: a rotating shaft that is mechanically connected to the lock mechanism and switches the state of the lock mechanism by rotating, and the indicator may include a base on which a display window is formed, a display plate to which a lock image indicating the locked state and a release image indicating the released state are added and which is configured to be movable between a lock position at which the lock image is exposed to an outside via the display window and a release position at which the release image is exposed to the outside via the display window, and a transmission mechanism that transmits rotational movement of the rotating shaft to the display plate to move the display plate between the lock position and the release position.

With such a configuration, the state of the lock mechanism and the display of the indicator can be linked in a simple configuration.

In this case, the transmission mechanism may include a biasing member that biases the display plate toward one of the lock position or the release position, and a pressing body that is mechanically connected to the rotating shaft, rotates together with the rotating shaft, and presses the display plate toward the other of the lock position or the release position against a biasing force of the biasing member in a case where the rotating shaft rotates in a forward direction or in a reverse direction.

With such a configuration, the rotation of the rotating shaft can be reliably transmitted to the display plate.

In addition, the indicator may include a lock sensor that detects a state of the lock mechanism and outputs an electric signal according to a detection result, and a display unit that switches a display content according to the electric signal from the lock sensor.

By using the electrical indicator, a degree of freedom in disposition of the indicator is improved.

In addition, the medical device may further comprise: a base having a predetermined color, the indicator may switch and display a lock image indicating the locked state and a release image indicating the released state in a display area defined in the base, a background color of the release image may be a color opposite to the color of the base, and a background color of the lock image may be a color similar to the color of the base.

With the above configuration, since the release image indicating releasing can be easily visually recognized even in a dark place, it is possible to effectively prevent “mistakenly recognizing that the drive caster is locked even though the lock is released”.

In this case, the lock image may include a background of a first color and a main figure that is a character or a figure drawn in a second color, the release image may include a background of the second color and a main figure that is a character or a figure drawn in the first color, and the first color may be a color opposite to the second color.

By configuring the release image and the lock image with two colors that are opposite to each other, the user can intuitively and clearly recognize which of the release image and the lock image is displayed.

In addition, the medical device may be an ultrasound diagnostic apparatus, the ultrasound diagnostic apparatus may further comprise a body part of the ultrasound diagnostic apparatus, one or more connection terminals that are provided on a front surface of the body part and to which a connector of an ultrasound probe is connected, a base that extends from a lower end of the body part to a front side, and an auxiliary light source that is provided near an upper end of the body part and illuminates the one or more connection terminals and the base, the indicator may have a display area in which a state of the lock mechanism is displayed, and the display area may be disposed on the base.

With such a configuration, the indicator is easily visible even in a dark place. In addition, by disposing the auxiliary light source, the connection terminal, and the indicator as described above, both the connection terminal and the indicator can be illuminated with one auxiliary light source.

With the medical device of the present disclosure, the user can more easily understand whether the drive caster is in the locked state or in the released state.

### BRIEF DESCRIPTION OF THE DRAWINGS

FIG. 1 is a perspective view of an ultrasound diagnostic apparatus.

FIG. 2 is a schematic diagram describing an operation of a lock mechanism.

FIG. 3 is a schematic exploded perspective view of a periphery of a display area.

FIG. 4A is a schematic diagram showing movement of a transmission mechanism.

FIG. 4B is a schematic diagram showing movement of the transmission mechanism.

FIG. 4C is a schematic diagram showing movement of the transmission mechanism.

FIG. 4D is a schematic diagram showing movement of the transmission mechanism.

FIG. 5 is a diagram showing an example of a release image and a lock image.

FIG. 6 is a schematic cross-sectional view of an indicator having an electrical element.

FIG. 7 is a schematic cross-sectional view of an indicator including a lock sensor and a display unit.

### DESCRIPTION OF THE EMBODIMENTS

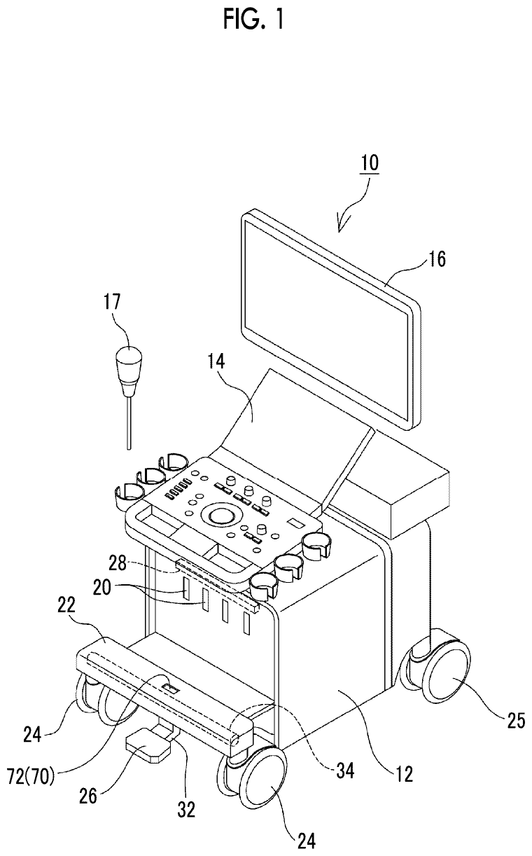

Hereinafter, a configuration of an ultrasound diagnostic apparatus 10 will be described with reference to the drawings. FIG. 1 is a schematic perspective view of the ultrasound diagnostic apparatus 10. The ultrasound diagnostic apparatus 10 is a device that transmits and receives ultrasound waves to and from a subject (for example, a living body) and that forms an ultrasound image, such as a tomographic image, based on a reception signal obtained by the transmission and reception, and is one type of medical devices.

The ultrasound diagnostic apparatus 10 includes a body part 12, an operation panel 14, and a display 16. The body part 12 is a main part of the ultrasound diagnostic apparatus 10, and serves as a base that supports the operation panel 14 and the display 16. Specifically, the body part 12 includes a controller (not shown) that controls an operation of the ultrasound diagnostic apparatus 10, a housing that accommodates the controller, and a resin-made exterior cover that covers the housing. A connection terminal 20 to which a connector of an ultrasound probe 17 is electrically connected is formed on a front surface of the body part 12.

The operation panel 14 receives an operation input from a user. The operation panel 14 includes, for example, various operation buttons, and a trackball. The display 16 displays the ultrasound image and various types of information. In the example of FIG. 1, the display 16 is a flat panel display such as a liquid crystal display. Both the operation panel 14 and the display 16 are attached to a multi-joint arm (not shown). Then, the user can freely change a position and a posture of the operation panel 14 and the display 16 by changing a posture of the multi-joint arm.

A plurality of drive casters 24 are attached to the body part 12. In the example of FIG. 1, two drive casters 24 are attached to a front part of the ultrasound diagnostic apparatus 10, and two drive casters 25 are attached to a rear part of the ultrasound diagnostic apparatus 10. Each of the drive casters 24 and 25 can rotate and turn. By providing the drive casters 24 and 25, a position of the ultrasound diagnostic apparatus 10 can be freely changed.

The ultrasound diagnostic apparatus 10 is further provided with a lock mechanism 30 that locks the movement of the drive caster 24 on the front side. One lock mechanism 30 is provided for each drive caster 24. The lock mechanism 30 can be switched between a locked state in which the movement of the drive caster 24 is locked and a released state in which the movement of the drive caster 24 is allowed. The state of the lock mechanism 30 is switched by operating a lock pedal 26 provided in a lower part of the ultrasound diagnostic apparatus 10.

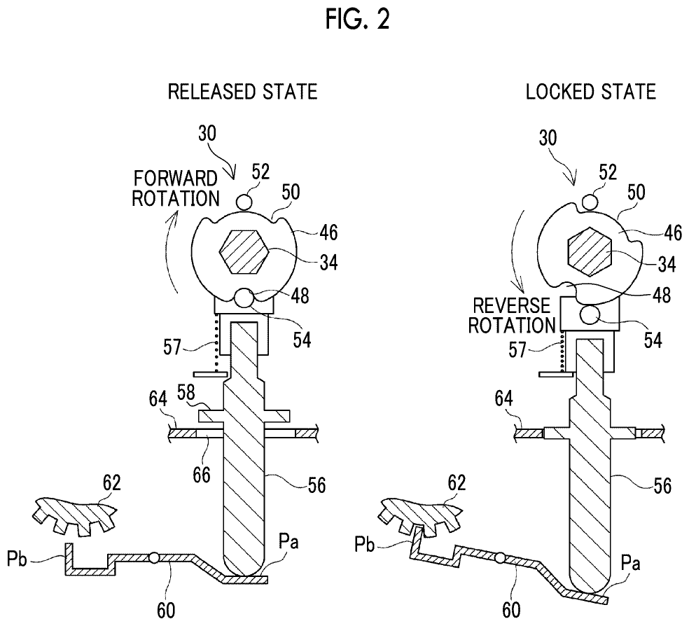

The lock mechanism 30 will be briefly described with reference to FIGS. 1 and 2. FIG. 2 is a schematic diagram describing an operation of the lock mechanism 30. As shown in FIG. 1, a base 22 that protrudes forward is connected to a lower part of the body part 12. The base 22 is a decorative panel made of a resin. A rotating shaft 34 extending in a vehicle width direction is disposed on a lower side of the base 22. The rotating shaft 34 is attached to the base 22 in a state of being rotatable about an axis parallel to the width direction. The rotating shaft 34 may be configured of a single shaft member or may be configured of a plurality of shaft members connected to each other. In any case, the rotating shaft 34 integrally rotates.

The lock pedal 26 is mechanically fixed to the rotating shaft 34 via a bracket 32. Therefore, the rotating shaft 34 also rotates in conjunction with the swing of the lock pedal 26. The swing of the lock pedal 26 is transmitted to the lock mechanism 30 via the rotating shaft 34.

FIG. 2 is a schematic diagram showing a configuration of the lock mechanism 30. The lock mechanism 30 includes a lock cam 46, a lifting shaft 56, a turning lock plate 58, a fixed plate 64, a rotation lock plate 60, and a lock gear 62. The lock cam 46 is a cam that is fixed to an end part of the rotating shaft 34 and rotates together with the rotating shaft 34. A cam groove 48 and a stopper groove 50 are formed on a peripheral surface of the lock cam 46. In a case where the rotating shaft 34 and the lock cam 46 are at angular positions shown in the left diagram of FIG. 2, the drive caster 24 enters a released state in which the rotation and turning of the drive caster 24 are allowed. Hereinafter, the angle of the rotating shaft 34 in the left diagram of FIG. 2 will be referred to as an “initial angle”. At the initial angle, the cam groove 48 is located directly below the rotating shaft 34 as viewed from a center of the rotating shaft 34.

The lifting shaft 56 is lifted and lowered with the rotation of the rotating shaft 34 and, by extension, with the rotation of the lock cam 46. Specifically, the lifting shaft 56 is biased upward by a spring 57. In addition, an abutment pin 54 protrudes from an upper peripheral surface of the lifting shaft 56. The abutment pin 54 abuts on the peripheral surface of the lock cam 46 by a biasing force of the spring 57.

As shown in the left diagram of FIG. 2, in a case where the rotating shaft 34 is at the initial angle, the abutment pin 54 enters the cam groove 48. On the other hand, as shown in the right diagram of FIG. 2, in a case where the rotating shaft 34 rotates from the initial angle, the lock cam 46 rotates to ride over the abutment pin 54. As a result, the abutment pin 54 and, by extension, the lifting shaft 56 are pressed downward. As a result, the lifting shaft 56 is lowered against the biasing force of the spring 57. In a case where the lifting shaft 56 is sufficiently lowered, the drive caster 24 enters a locked state in which the rotation and turning of the drive caster 24 are prohibited. Hereinafter, the angle of the rotating shaft 34 in the right diagram of FIG. 2 will be referred to as a “lock angle”. In addition, hereinafter, the rotation in a direction in which the angle of the rotating shaft 34 approaches the lock angle from the initial angle is referred to as “forward rotation”, and the rotation in a direction in which the angle of the rotating shaft 34 approaches the initial angle from the lock angle is referred to as “reverse rotation”.

The stopper pin 52 is located inside the stopper groove 50 of the lock cam 46 and is positionally invariable. As the rotating shaft 34 rotates, the stopper pin 52 abuts on a circumferential end part of the stopper groove 50, so that further rotation of the rotating shaft 34 is restricted.

The fixed plate 64 is a plate that turns together with the drive caster 24. A lock hole 66 into which the turning lock plate 58 can enter is formed in the fixed plate 64. The turning lock plate 58 is a plate that is fixed to the peripheral surface of the lifting shaft 56 and that can be lifted and lowered together with the lifting shaft 56. In a case where the rotating shaft 34 is at the initial angle, the turning lock plate 58 is located above the lock hole 66. On the other hand, in a case where the rotating shaft 34 is at the lock angle, the turning lock plate 58 is located inside the lock hole 66 as shown in the right diagram of FIG. 2. In a case where the turning lock plate 58 enters the lock hole 66, the turning lock plate 58 and the lock hole 66 interfere with each other, so that the turning of the drive caster 24 is restricted.

The lock gear 62 is a gear that is fixed to the drive caster 24 and that rotates together with the drive caster 24. The rotation lock plate 60 is a plate that is disposed on a lower side of the lifting shaft 56 and that swings about a shaft that is positionally invariable. One end of the rotation lock plate 60 functions as a force point Pa in contact with the lifting shaft 56, and the other end functions as an action point Pb that engages with the lock gear 62.

As shown in the left diagram of FIG. 2, in a case where the rotating shaft 34 is at the initial angle, the action point Pb of the rotation lock plate 60 is completely separated from the lock gear 62, and the lock gear 62 and the drive caster 24 can be freely rotated. On the other hand, as shown in the right diagram of FIG. 2, in a case where the rotating shaft 34 is at the lock angle, the action point Pb of the rotation lock plate 60 engages with the lock gear 62. In this case, the rotation of the lock gear 62 and the drive caster 24 is prohibited.

As is clear from the above description, the rotating shaft 34 is mechanically connected to the lock mechanism 30, and the rotation and turning of the drive caster 24 are locked or unlocked with the rotation of the rotating shaft 34. Such a configuration of the lock mechanism 30 is an example, and the configuration of the lock mechanism 30 may be appropriately changed as long as the locked state and the released state are switched with the rotation of the rotating shaft 34.

Incidentally, as is clear from the description so far, the state of the lock mechanism 30 is switched by stepping on or releasing the lock pedal 26. Therefore, the state of the lock mechanism 30 can be understood to some extent by visually recognizing the angle of the lock pedal 26. However, in order to understand the state of the lock mechanism 30 only with the angle of the lock pedal 26, the user needs to accurately understand a correspondence relationship between the angle of the lock pedal 26 and the state of the lock mechanism 30 in advance.

Meanwhile, a certain degree of familiarity is required for the user to understand such a correspondence relationship. In addition, even in a case where the user is familiar to the operation to some extent, it is difficult to intuitively understand the state of the lock mechanism 30 even by looking at the angle of the lock pedal 26. As a result, in the related art, the user may take time to understand the state of the lock mechanism 30 or may mistakenly recognize the state of the lock mechanism 30.

Therefore, the ultrasound diagnostic apparatus 10 of the present disclosure includes an indicator 70 that presents the state of the lock mechanism 30 to the user. The indicator 70 has a display area 72 in which the state of the lock mechanism 30 is displayed. In the example of FIG. 1, the display area 72 is disposed near the center of the base 22 in the width direction. In other words, the display area 72 is disposed upward at a position adjacent to the lock pedal 26 in a front-rear direction. With such a disposition, in a case where the user looks at the lock pedal 26 in order to operate the lock pedal 26, the display area 72 is also likely to come within a field of vision simultaneously. As a result, the user can easily understand the current state of the lock mechanism 30 before operating the lock pedal 26.

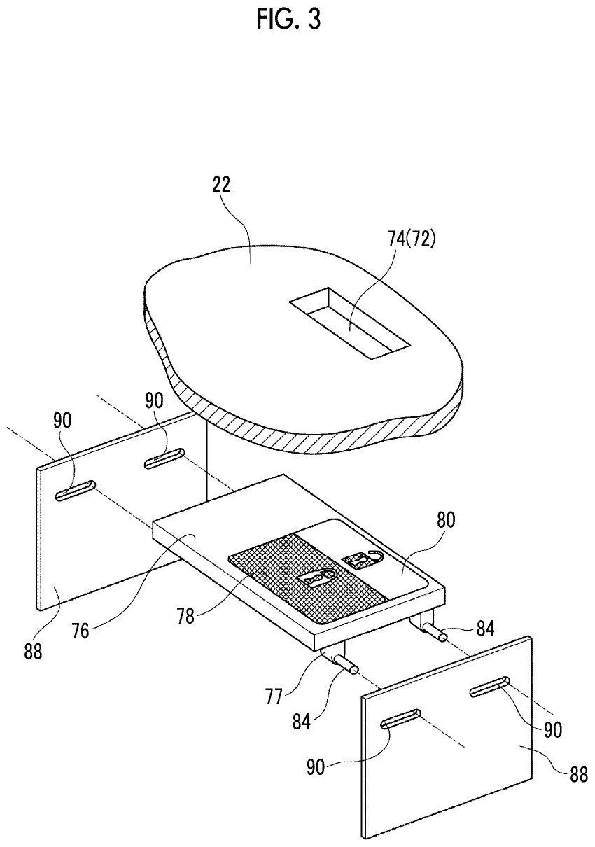

FIG. 3 is a schematic exploded perspective view of a periphery of the display area 72. As shown in FIG. 3, a display window 74, which is a substantially rectangular through-hole, is formed in the base 22. The display window 74 functions as the display area 72 of the indicator 70.

A display plate 76 is disposed on the lower side of the base 22. The display plate 76 is attached to the ultrasound diagnostic apparatus 10 in a state of being slidable in the front-rear direction. More specifically, a plurality of ribs 77 are erected from a bottom surface of the display plate 76. A guide pin 84 protrudes from an outer end part of the rib 77 in the width direction.

A pair of support plates 88 are disposed on both sides of the display plate 76 in the width direction. The support plate 88 is fixed to the base 22. In addition, a long guide hole 90 is formed in the support plate 88 in the front-rear direction. The guide pin 84 is inserted into the guide hole 90. As a result, the display plate 76 is guided to move in the front-rear direction.

Two images showing the state of the lock mechanism 30, that is, a lock image 78 and a release image 80 are drawn side by side on an upper surface of the display plate 76 in the front-rear direction. The lock image 78 is an image indicating that the lock mechanism 30 is in a locked state. In the example of FIG. 3, the lock image 78 includes a pictogram of a locked key. The release image 80 is an image indicating that the lock mechanism 30 is in a released state. In the example of FIG. 3, the release image 80 includes a pictogram of an unlocked key.

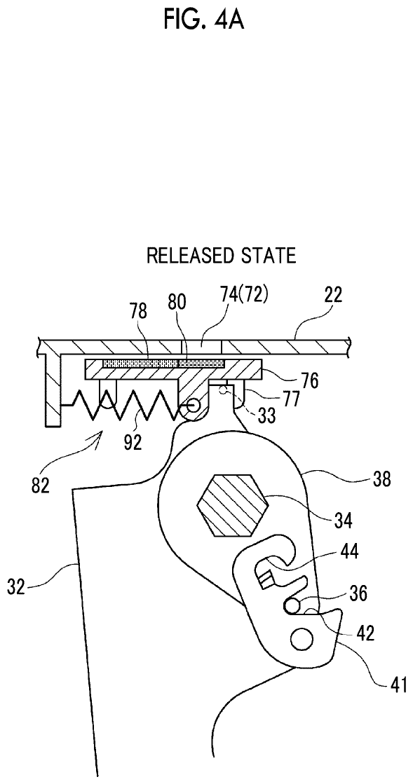

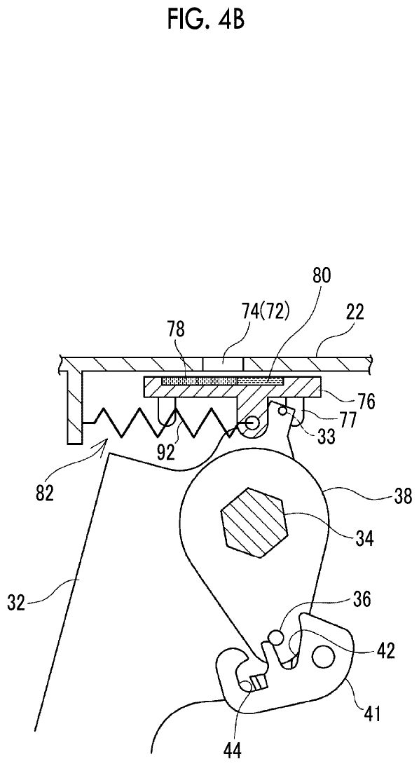

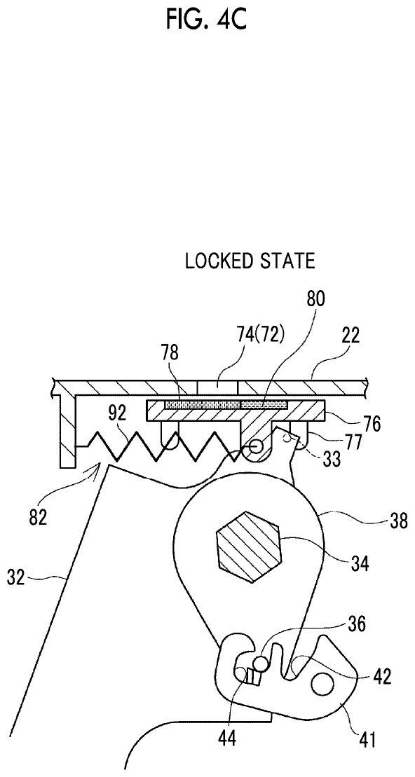

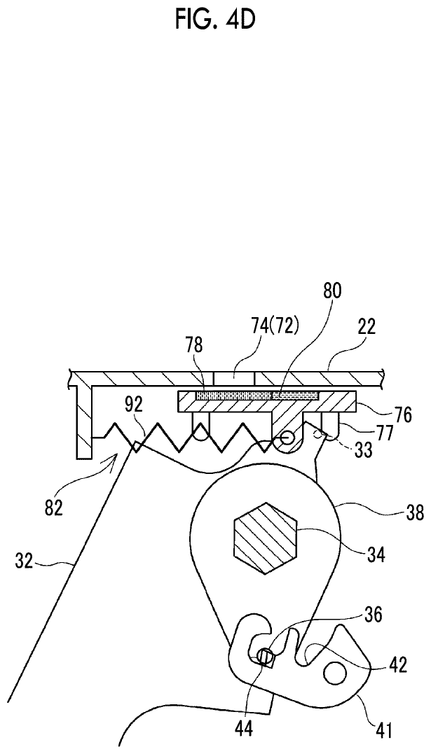

The indicator 70 includes a transmission mechanism 82 that slidably moves the display plate 76 in conjunction with the rotation of the rotating shaft 34. FIGS. 4A to 4D are schematic diagrams showing movement of the transmission mechanism 82. FIG. 4A to FIG. 4D are cross-sectional views of the vicinity of the indicator 70 taken along a plane orthogonal to the width direction.

As described above, the lock pedal 26 is connected to the rotating shaft 34 via the bracket 32, and the lock pedal 26, the bracket 32, and the rotating shaft 34 integrally rotate. The transmission mechanism 82 converts the rotational movement of the rotating shaft 34 into linear movement and transmits the linear movement to the display plate 76. The transmission mechanism 82 includes a pressing body 33 fixed to the bracket 32, and a spring for sliding 92. The pressing body 33 is located at a position aligned with the rib 77 of the display plate 76 in the front-rear direction, and presses the rib 77 and, by extension, the display plate 76 rearward (that is, in a right direction of the paper plane in FIG. 4).

The spring for sliding 92 biases the display plate 76 forward (that is, in a left direction of the paper plane in FIG. 4). One end of the spring for sliding 92 is attached to the base 22, and the other end is attached to the display plate 76.

FIG. 4A shows a situation in a case where the rotating shaft 34 is at the initial angle, that is, in a case where the lock mechanism 30 is in the released state. In this case, the display plate 76 is located on the front side (that is, the left side of the paper plane) by a biasing force of the spring for sliding 92. In this case, the release image 80 of the display plate 76 is located directly below the display window 74. Therefore, in this case, the user can visually recognize the release image 80 via the display window 74. Hereinafter, the position of the display plate 76 in a case where the rotating shaft 34 is at the initial angle is referred to as a “release position”.

Thereafter, the lock pedal 26 is operated, and the rotating shaft 34 gradually rotates in the forward direction from the initial angle. In this case, as shown in FIG. 4B, the pressing body 33 provided at an upper part of the bracket 32 gradually moves rearward. The display plate 76 also moves rearward by being pressed by the pressing body 33. Then, in a case where the rotating shaft 34 rotates to the lock angle, as shown in FIG. 4C, the lock image 78 is located directly below the display window 74. As a result, the user can visually recognize the lock image 78 via the display window 74. Hereinafter, the position of the display plate 76 in a case where the rotating shaft 34 is at the lock angle is referred to as a “lock position”.

The rotating shaft 34 is latched at the initial angle and the lock angle. In order to enable this latching, a latch plate 38 that rotates together with the rotating shaft 34 is further fixed to the rotating shaft 34. The latch plate 38 is provided with a latch pin 36. In addition, a latch cam 41 that swings about a predetermined support shaft 40 is attached to the support plate 88. The latch cam 41 is biased in a direction of an arrow A by a spring (not shown). In addition, an outer shape of the latch cam 41 is largely wavy, and, by this wavy shape, a first latch groove 42 and a second latch groove 44 into which the latch pin 36 is fitted are formed. The latch pin 36 is fitted into the first latch groove 42 or the second latch groove 44 with the rotation of the rotating shaft 34, whereby the released state or the locked state is maintained. In the locked state (that is, in the state of FIG. 4C), in a case where the lock pedal 26 is stepped on, the latch is released, and as shown in FIG. 4D, the rotating shaft 34 temporarily rotates further in the forward direction from the lock angle. Then, in a case where the latch is completely released, the rotating shaft 34 rotates to the initial angle in the reverse direction.

As is clear from the above description, according to this example, the display plate 76 slidably moves in conjunction with the rotation of the rotating shaft 34. Then, the image displayed on the display window 74 is switched. As a result, the user can intuitively understand the state of the lock mechanism 30 by visually recognizing the image displayed on the display window 74.

In addition, in a case of this example, the rotation of the rotating shaft 34 is mechanically transmitted to the display plate 76 via the transmission mechanism 82. Therefore, the position of the display plate 76 can be reliably and accurately synchronized with the state of the rotating shaft 34 and, by extension, the state of the lock mechanism 30. In other words, it is possible to reliably prevent a problem in that the display of the indicator 70 does not match the state of the lock mechanism 30.

In addition, as is clear from the description so far, in this example, the display of the indicator 70 is switched only by the mechanical mechanism. In other words, the above-described indicator 70 does not require an electrical configuration. As a result, the overall configuration of the indicator 70 can be simplified. In addition, in this case, the indicator 70 can be appropriately switched even in a state in which the power of the ultrasound diagnostic apparatus 10 is turned OFF.

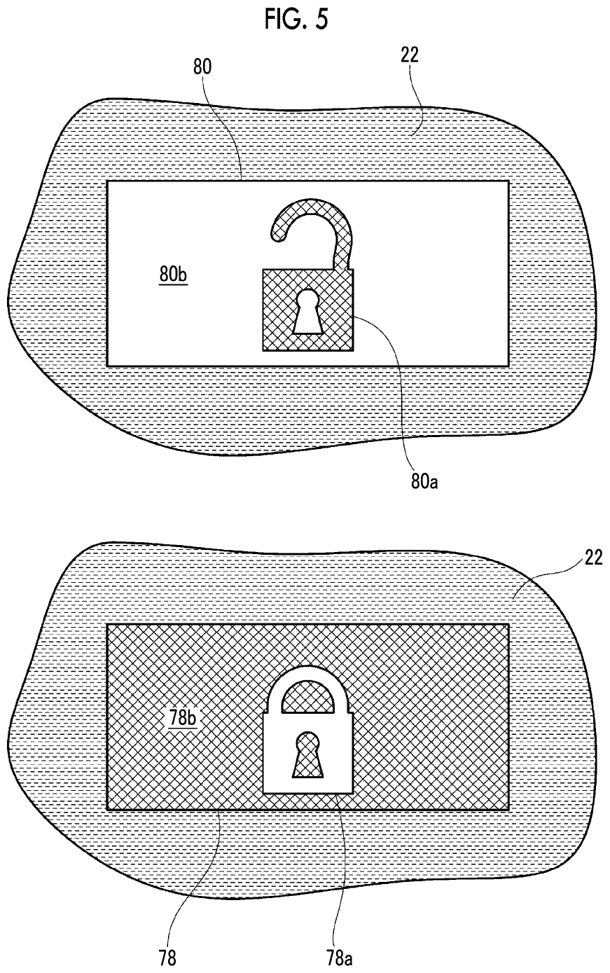

Next, the release image 80 and the lock image 78 drawn on the display plate 76 will be described. FIG. 5 is a diagram showing an example of the release image 80 and the lock image 78. The release image 80 includes a main FIG. 80a and a background 80b. The background 80b is a plain region that does not include a line or a figure. The main FIG. 80a is a part other than the background 80b and is composed of a character or a figure. In the example of FIG. 5, a pictogram of an unlocked key corresponds to the main FIG. 80a. Similarly, the lock image 78 also includes a main FIG. 78a and a background 78b. In the example of FIG. 5, a pictogram of a locked key corresponds to the main FIG. 78a.

Here, in a case where a color of the main FIG. 80a of the release image 80 is “first color” and a color of the background 80b is a “second color”, the main FIG. 78a of the lock image 78 is the second color, and the background 78b is the first color. That is, in the release image 80 and the lock image 78, the colors of the main figures are inverted from each other, and the colors of the backgrounds are inverted from each other. In addition, the second color is a color opposite to the first color. With such a configuration, the user can clearly distinguish between the release image 80 and the lock image 78, and can more intuitively understand the state of the lock mechanism 30. The opposite color is a color having an opposing property. For example, the opposite color is a color in which at least one of a hue, a brightness, or a chroma saturation is opposed.

In addition, in this example, the second color (that is, the color of the background 80b of the release image 80) has a higher brightness than the first color (that is, the color of the background 78b of the lock image 78). For example, the first color is black and the second color is white. The reason for such a configuration will be described.

First, a case is considered in which the user mistakenly recognizes that the lock mechanism 30 is in the released state even though the lock mechanism 30 is actually in the locked state. In this case, even though the user tries to move the ultrasound diagnostic apparatus 10, the ultrasound diagnostic apparatus 10 does not move, but this itself is not a significant problem. On the other hand, a case is considered in which the user mistakenly recognizes that the lock mechanism 30 is in the locked state even though the lock mechanism 30 is actually in the released state. In this case, the user may apply a force to the ultrasound diagnostic apparatus 10 assuming that the ultrasound diagnostic apparatus 10 does not move, which may cause the ultrasound diagnostic apparatus 10 to move unexpectedly. Such unintentional movement of the ultrasound diagnostic apparatus 10 causes various problems. Therefore, in a case where the lock mechanism 30 is in the released state, in the present example, in order to reliably inform the user of the released state, the color of the background 80b of the release image 80 is set to a higher brightness than the color of the background 78b of the lock image 78, so that it is noticeable even in a dark environment.

In addition, in this example, the second color (that is, the color of the background 80b of the release image 80) is a color opposite to the color of the base 22. For example, in a case of the example of FIG. 5, the base 22 is gray, that is, a color similar to the first color, and the second color is white. With such a configuration, the visibility of the release image 80 is further improved. As a result, it is possible to effectively prevent mistakenly recognizing that the lock mechanism 30 is in the locked state even though the lock mechanism 30 is actually in the released state.

It is needless to say that the forms of the release image 80 and the lock image 78 described so far may be changed as appropriate. For example, in the above description, the main FIGS. 80a and 78a are used as the key pictograms, but the main FIGS. 80a and 78a may be other figures or characters. In addition, the combination of the colors of the main FIGS. 80a and 78a and the backgrounds 80b and 78b may also be changed as appropriate.

In addition, not only the forms of the release image 80 and the lock image 78 but also other configurations may be changed as appropriate. For example, in the description so far, the display area 72 of the indicator 70 is provided near the lock pedal 26. However, the display area 72 may be provided at another location. In this case, the display area 72 may be disposed within a width of the rotating shaft 34. With such a configuration, the rotation of the rotating shaft 34 can be transmitted to the display area 72 with a simple configuration. In addition, the display area 72 is not limited to facing directly upward, and may be directed in another direction. For example, the display area 72 may be disposed in a posture obliquely facing an upper front direction toward a front upper side. Even in such a posture, the user can easily visually recognize the display area 72.

In addition, the ultrasound diagnostic apparatus 10 may be further provided with an auxiliary light source 28 (see FIG. 1) that illuminates the connection terminal 20. The auxiliary light source 28 may be disposed, for example, on the front surface and in the vicinity of an upper end of the body part 12. The ultrasound diagnostic apparatus 10 is often used in a dark place, but by providing the auxiliary light source 28, the user can clearly visually recognize the connection terminal 20 even in the dark place. In addition, in this example, the display area 72 is provided on the base 22 that extends from a lower end of the body part 12 to the front side. With this, the auxiliary light source 28 can illuminate not only the connection terminal 20 but also the display area 72. As a result, the visibility of the display area 72 can also be improved by providing the auxiliary light source 28 that illuminates the connection terminal 20.

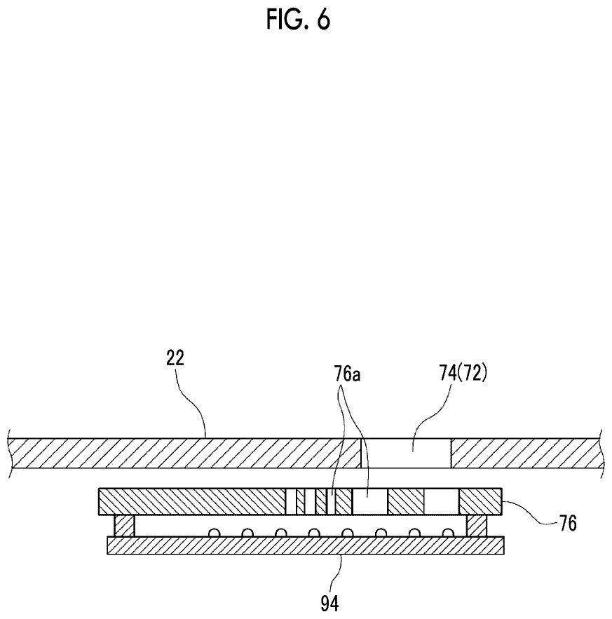

In addition, in the description so far, the indicator 70 is composed of only mechanical elements. However, the indicator 70 may have an electrical element as necessary. For example, a light source 94 may be disposed below the display plate 76 to illuminate the display plate 76 from below. FIG. 6 is a schematic cross-sectional view of the display plate 76 in this case. As shown in FIG. 6, in this case, a light guide path 76a through which light passes is partially or entirely formed in the display plate 76. The light guide path 76a may be, for example, a through-hole or a translucent material (for example, a transparent resin) embedded in the through-hole.

In addition, the light guide path 76a may be formed by locally thinning the display plate 76 to the extent that light can be seen through. Further, the entire display plate 76 may be made of a translucent material, thereby allowing the entire display plate 76 to function as the light guide path 76a.

The light source 94 is disposed behind the display plate 76. The light source 94 is not particularly limited, and for example, the light source 94 may be an LED substrate on which one or more LED elements are mounted. In a case where the light source 94 is turned on, the lock image 78 and the release image 80 of the display plate 76 are illuminated. As a result, the visibility of the indicator 70 in a dark place can be further improved.

The power may be supplied to the light source 94 by a power supply device of the ultrasound diagnostic apparatus 10, or a battery (for example, a dry battery) independent of the ultrasound diagnostic apparatus 10 may be provided for the light source 94. By providing the independent battery, the indicator 70 can be appropriately illuminated even in a case where the power of the ultrasound diagnostic apparatus 10 is turned OFF.

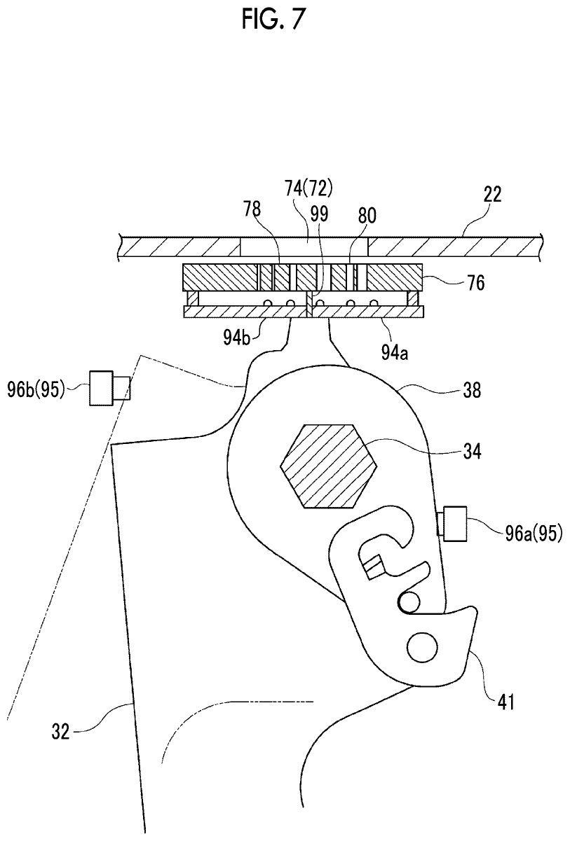

In addition, as another aspect, the indicator 70 may include a lock sensor 95 that detects a state of the lock mechanism 30 and outputs an electric signal according to a detection result, and a display unit 98 that switches a display content according to the electric signal from the lock sensor 95. The lock sensor 95 may be, for example, an encoder that detects a rotation angle of the rotating shaft 34. In addition, as another aspect, the lock sensor 95 may be a switch that is turned ON in a case where the rotating shaft 34 reaches a predetermined rotation angle. For example, as shown in FIG. 7, the lock sensor 95 may include a first switch 96a and a second switch 96b. In this case, the first switch 96a is disposed at a position where the first switch 96a is turned ON by coming into contact with a mechanical element (latch plate 38 in the illustrated example) that moves in conjunction with the rotation of the rotating shaft 34 in the released state. In addition, the second switch 96b is disposed at a position where the second switch 96b is turned ON by coming into contact with a mechanical element (lock pedal 26 in the illustrated example) that moves in conjunction with the rotation of the rotating shaft 34 in the locked state.

The display unit 98 is not particularly limited as long as it can switch the display content according to an electrical signal. For example, the display unit 98 may include a display that switches the display content according to the output signal from the lock sensor 95. In addition, as another aspect, the display unit 98 may include a plurality of light sources 94a and 94b of which a lighting state is switched according to the output signal from the lock sensor 95. For example, as shown in FIG. 7, the display unit 98 may include the display plate 76, the first light source 94a that illuminates the release image 80, and the second light source 94b that illuminates the lock image 78. In this case, the first light source 94a is disposed on a back side of the release image 80, and the second light source 94b is disposed on a back side of the lock image 78. In addition, a light screen 99 is disposed between the first light source 94a and the second light source 94b.

The first light source 94a is turned on only in a case where the first switch 96a is turned ON, and the second light source 94b is turned on only in a case where the second switch 96b is turned ON. As a result, the appropriate images 78 and 80 are illuminated according to the state of the lock mechanism 30, so that the user can more easily understand the state of the lock mechanism 30.

In this case, the display plate 76 does not slidably move and is positionally invariable with respect to the display window 74. In that case, the display window 74 has a size such that both the lock image 78 and the release image 80 are exposed to the outside. With such a configuration, since the mechanism for moving the display plate 76 is unnecessary, the mechanical configuration around the rotating shaft 34 can be simplified.

Incidentally, in this case, it is not necessary to mechanically transmit the rotational movement of the rotating shaft 34 to the display unit 98 including the display plate 76, and it is not necessary to dispose the display unit 98 in the vicinity of the rotating shaft 34. Therefore, in this case, the display unit 98 may be disposed at a location away from the rotating shaft 34, for example, on the operation panel 14.

In addition, in the description so far, the indicator 70 incorporated in the ultrasound diagnostic apparatus 10 has been described as an example. However, the indicator 70 of the present disclosure is not limited to the ultrasound diagnostic apparatus 10, and may be incorporated into another caster moving type medical device. For example, the indicator 70 may be applied to a caster moving type X-ray diagnostic apparatus or the like.

### EXPLANATION OF REFERENCES

What is claimed is:

1. A medical device comprising:
a plurality of drive casters;
a plurality of lock mechanisms that are respectively provided in the plurality of drive casters, each of the plurality of lock mechanisms locking movement of a corresponding drive caster; and
an indicator that presents whether the lock mechanism is in a locked state in which the movement of the drive caster is locked or in a released state in which the movement of the drive caster is allowed.

2. The medical device according to claim 1,
wherein the indicator has a display area in which a state of the lock mechanism is displayed, and
the display area is disposed upward or disposed in a posture obliquely facing an upper front direction toward a front upper side, at a lower front part of the medical device.

3. The medical device according to claim 2, further comprising:
a rotating shaft that switches the state of the lock mechanism by rotating,
wherein the display area is disposed within a width of the rotating shaft.

4. The medical device according to claim 1, further comprising:
a rotating shaft that is mechanically connected to the lock mechanism and switches a state of the lock mechanism by rotating,
wherein the indicator includes
a base on which a display window is formed,
a display plate to which a lock image indicating the locked state and a release image indicating the released state are added and which is configured to be movable between a lock position at which the lock image is exposed to an outside via the display window and a release position at which the release image is exposed to the outside via the display window, and
a transmission mechanism that transmits rotational movement of the rotating shaft to the display plate to move the display plate between the lock position and the release position.

5. The medical device according to claim 4,
wherein the transmission mechanism includes
a biasing member that biases the display plate toward one of the lock position or the release position, and
a pressing body that is mechanically connected to the rotating shaft, rotates together with the rotating shaft, and presses the display plate toward the other of the lock position or the release position against a biasing force of the biasing member in a case where the rotating shaft rotates in a forward direction or in a reverse direction.

6. The medical device according to claim 1,
wherein the indicator includes
a lock sensor that detects a state of the lock mechanism and outputs an electric signal according to a detection result, and
a display unit that switches a display content according to the electric signal from the lock sensor.

7. The medical device according to claim 1, further comprising:
a base having a predetermined color,
wherein the indicator switches and displays a lock image indicating the locked state and a release image indicating the released state in a display area defined in the base,
a background color of the release image is a color opposite to the color of the base, and
a background color of the lock image is a color similar to the color of the base.

8. The medical device according to claim 7,
wherein the lock image includes a background of a first color and a main figure that is a character or a figure drawn in a second color,
the release image includes a background of the second color and a main figure that is a character or a figure drawn in the first color, and
the first color is a color opposite to the second color.

9. The medical device according to claim 1,
wherein the medical device is an ultrasound diagnostic apparatus,
the ultrasound diagnostic apparatus further comprises
a body part of the ultrasound diagnostic apparatus,
one or more connection terminals that are provided on a front surface of the body part and to which a connector of an ultrasound probe is connected,
a base that extends from a lower end of the body part to a front side, and
an auxiliary light source that is provided near an upper end of the body part and illuminates the one or more connection terminals and the base,
the indicator has a display area in which a state of the lock mechanism is displayed, and
the display area is disposed on the base.
